(19) United States
(12) Patent Application Publication
(10) Pub. No.: US 2009/0047245 A1
(43) Pub. Date: Feb. 19, 2009

(54) Prognostic chronic hepatitis C biomarkers

(76) Inventor: Zobair M. Younossi, Fairfax Station, VA (US)

Correspondence Address: MCDERMOTT WILL & EMERY LLP, 600 13TH STREET, N.W., WASHINGTON, DC, 20005-3096, US

(21) Appl. No.: 12/155,562

(22) Filed: Jun. 5, 2008

Related U.S. Application Data
(60) Provisional application No. 60/942,376, filed on Jun. 6, 2007.
(60) Provisional application No. 60/935,863, filed on Sep. 5, 2007.

(51) Int. Cl. A61K 38/21; C12Q 1/68; A61K 31/7105

(52) U.S. Cl. 424/85.4; 435/6; 514/44

(57) ABSTRACT

Methods for identifying biomarkers useful in determining the response of hepatitis C virus (HCV) infected patients hepatitis C specific treatment. In particular, the providing biomarkers that can be used to identify sustained responders and non-responders prior to and/or during hepatitis C specific treatment.

### CROSS REFERENCE TO RELATED APPLICATION

This application claims the benefit of U.S. Provisional Application U.S. Ser. No. 60/942,376, filed on Jun. 6, 2007, and U.S. Provisional Application U.S. No. 60/935,863, filed Sep. 5, 2007, which is incorporated herein by reference.

### BACKGROUND OF THE INVENTION

1. Field of the Invention

The present invention relates to the identification of biomarkers useful in determining the response of hepatitis C virus (HCV) infected patients hepatitis C specific treatment. In particular, the invention provides biomarkers that can be used to identify sustained responders and non-responders prior to and/or during hepatitis C specific treatment.

2. Background

The hepatitis C virus (HCV) is one of the most important causes of chronic liver disease in the United States. It accounts for about 15 percent of acute viral hepatitis, 60 to 70 percent of chronic hepatitis, and up to 50 percent of cirrhosis, end-stage liver disease, and liver cancer. Of the U.S. population, 1.6 percent, or an estimated 4.1 million Americans, have antibody to HCV (anti-HCV), indicating ongoing or previous infection with the virus. Hepatitis C causes an estimated 10,000 to 12,000 deaths annually in the United States.

A distinct and major characteristic of hepatitis C is its tendency to cause chronic liver disease in which the liver injury persists for a prolonged period if not for life. About 75 percent of patients with acute hepatitis C ultimately, develop chronic infection. This exceedingly high rate of chronicity is due to the ability of the virus to undergo high rates of mutation resulting in different genotypes and the co-existence of “escape mutants” as a quasi-species.

Chronic hepatitis C varies greatly in its course and outcome. At one end of the spectrum are infected persons who have no signs or symptoms of liver disease and have completely normal levels of serum enzymes, the usual blood test results that indicate liver disease. Liver biopsy usually shows some degree of injury to the liver, but the extent is usually mild, and the overall prognosis may be good. At the other end of the spectrum are patients with severe hepatitis C who have symptoms, high levels of the virus (HCV RNA) in serum, and elevated serum enzymes, and who ultimately develop cirrhosis and end-stage liver disease. In the middle of the spectrum are many patients who have few or no symptoms, mild to moderate elevations in liver enzymes, and an uncertain prognosis.

Chronic hepatitis C can cause cirrhosis, liver failure, and liver cancer. Researchers estimate that at least 20 percent of patients with chronic hepatitis C develop cirrhosis, a process that takes at least 10 to 20 years. Liver failure from chronic hepatitis C is one of the most common reasons for liver transplants in the United States. After 20 to 40 years, a small percentage of patients develop liver cancer. Hepatitis C is the cause of about half of cases of primary liver cancer in the developed world. Men, alcoholics, patients with cirrhosis, people over age 40, and those infected for 20 to 40 years are at higher risk of developing HCV-related liver cancer.

HCV is a small (40 to 60 nanometers in diameter), enveloped, single-stranded RNA virus of the family Flaviviridae and genus hepacivirus. Because the virus mutates rapidly, changes in the envelope proteins may help it evade the immune system. There are at least six major genotypes and more than 50 subtypes of HCV. The different genotypes have different geographic distributions. Genotypes 1a and 1b are the most common in the United States (about 75 percent of cases). Genotypes 2 and 3 are present in only 10 to 20 percent of patients. There is little difference in the severity of disease or outcome of patients infected with different genotypes. However, patients with genotypes 2 and 3 are more likely to respond to treatment.

Interferon (IFN) based regimens form the backbone of anti-viral therapy against HCV. The mechanism by which interferon acts is not well understood. Interferon may have both direct antiviral (by inducing enzymes that interfere with the production of viral proteins and their subsequent replication) and immunomodulatory effects (by enhancing the HLA-I restricted cellular immunity and viral clearance).

Trials of interferon (3 million units (MU) three times/week (T.I.W.) for 12 months) for chronic HCV infection have resulted in 15-20% sustained virologic response (‘SVR’) rates. The therapy for chronic hepatitis C has evolved steadily since alpha interferon was first approved for use in this disease more than 10 years ago.

Alpha interferon is a host protein that is made in response to viral infections and has natural antiviral activity. Recombinant forms of alpha interferon have been produced, and several formulations (alfa-2a, alfa-2b, consensus interferon) are available as therapy for hepatitis C.

Recent studies of the effect of interferon on the kinetics of HCV suggest that viral suppression can be achieved more efficiently if interferon is delivered in a more sustained fashion. Additionally, fluctuating levels of viremia, which occur with TIW dosing, probably contribute to the ability of HCV to develop “escape mutants” and become resistant to treatment.

Peginterferon is alpha interferon that has been modified chemically by the addition of a large inert molecule of polyethylene glycol. Pegylation changes the uptake, distribution, and excretion of interferon, prolonging its half-life. Peginterferon can be given once weekly and provides a constant level of interferon in the blood, whereas standard interferon must be given several times weekly and provides intermittent and fluctuating levels. In addition, peginterferon is more active than standard interferon in inhibiting HCV and yields higher sustained response rates with similar side effects. Because of its ease of administration and better efficacy, peginterferon has replaced standard interferon both as monotherapy and as combination therapy for hepatitis C. Promising new longer-acting forms of interferon alpha, e.g., Albuferon, have also being created using albumin-fusion technology.

Ribavirin is an oral antiviral agent that has activity against a broad range of viruses. By itself, ribavirin has little effect on HCV, but adding it to interferon increases the sustained response rate by two- to three-fold. For these reasons, combination therapy is now recommended for hepatitis C, and interferon monotherapy is applied only when there are specific reasons not to use ribavirin.

Combination therapy leads to rapid improvements in serum ALT levels and disappearance of detectable HCV RNA in up to 70 percent of patients. However, long-term improvement in hepatitis C occurs only if HCV RNA disappears during therapy and stays undetectable once therapy is stopped. Among patients who become HCV RNA negative during treatment, some will relapse when therapy is stopped. The relapse rate is lower in patients treated with combination therapy compared with monotherapy. Thus, a 48-week course of combination therapy using peginterferon and ribavirin yields a sustained response rate of about 55 percent. A similar course of peginterferon monotherapy yields a sustained response rate of only 35 percent. A response is considered “sustained” if HCV RNA remains undetectable for 6 months or more after stopping therapy.

At the present time, the optimal regimen appears to be a 24- or 48-week course of the combination of pegylated alpha interferon and ribavirin based on HCV genotype. This therapy is expensive and costs approximately $18,000 per year per patient.

Common side effects of alpha interferon and peginterferon (occurring in more than 10 percent of patients) include fatigue, muscle aches, headaches, nausea and vomiting, skin irritation at the injection site, low-grade fever, weight loss, irritability, depression, mild bone marrow suppression and hair loss. Less common side effects of alpha interferon, peginterferon, and combination therapy include autoimmune disease (especially thyroid disease), severe bacterial infections, marked thrombocytopenia, marked neutropenia, seizures, depression and suicidal ideation or attempts, retinopathy (microhemorrhages), hearing loss, acute congestive heart failure, renal failure, vision loss, pulmonary fibrosis or pneumonitis, and sepsis. Deaths have been reported from acute myocardial infarction, stroke, suicide, and sepsis

Ribavirin also causes side effects, and the combination is generally less well tolerated than peginterferon monotherapy. The most common side effects of ribavirin are anemia, fatigue and irritability, itching, skin rash, nasal stuffiness, sinusitis and cough.

Accordingly, many attempts have been made to identify patients who have a rapid response to treatment and who might be able to stop peginterferon and ribavirin early and be spared the further expense and side effects of prolonged therapy. The consequence of early stopping, however, is a higher relapse rate and this approach of abbreviating therapy in rapid responders must be individualized based upon tolerance.

To date, there are no reliable prognostic biomarkers available for predicting whether or not a hepatitis C patient will or will not respond to therapy. Such a biomarker would greatly lower side effects, reduce costs associated with hepatitis C treatment on health care systems, as well as increase the efficacy of therapy and the quality of life for millions of patients.

### SUMMARY OF THE INVENTION

One aspect of the invention relates to a method of treating hepatitis C in a hepatitis C treatment-naïve or hepatitis C treatment non responsive patient comprising obtaining a biological sample from the patient; isolating total mRNA from the biological sample; determining an amount of marker gene mRNA transcribed from a gene selected from the group consisting of those listed in Table 1 relative to an amount of one or more internal control mRNAs selected from the group of Table 2, present in the total mRNA to obtain a relative marker gene index; providing the patient with a hepatitis C-specific therapeutic agent when the relative marker gene index is above a threshold level that correlates with sustained viroloic response (SVR).

In one embodiment, the biological sample is obtained prior to the patient receiving a hepatitis C-specific therapeutic agent. In another embodiment, the biological sample is obtained about 24 hours after treatment with a hepatitis C-specific therapeutic agent is initiated. In a further embodiment, the biological sample is obtained about 7 days after treatment with a hepatitis C-specific therapeutic agent is initiated. In yet another embodiment, the biological sample is obtained about 28 days after treatment with a hepatitis C-specific therapeutic agent is initiated. In still another embodiment, the biological sample is obtained about 56 days after treatment with a hepatitis C-specific therapeutic agent is initiated.

In still another embodiment, the hepatitis C is genotype 1 hepatitis C. In a further embodiment, the biological sample is blood. In still another embodiment, the hepatitis C-specific therapeutic agent comprises ribivirin. In another embodiment, the hepatitis C-specific therapeutic agent comprises an interferon. In yet a further embodiment, the interferon is interferon alpha. In still another, the interferon is pegylated interferon alpha. In yet another, the interferon is interferon fused to albumin. In still a further embodiment, the hepatitis C-specific therapeutic agent comprises ribivirin and interferon. In another embodiment, the hepatitis in chronic hepatitis.

Additional advantages of the present invention will become readily apparent to those skilled in this art from the following detailed description, wherein only the preferred embodiment of the invention is shown and described, simply by way of illustration of the best mode contemplated of carrying out the invention. As will be realized, the invention is capable of other and different embodiments, and its several details are capable of modifications in various obvious respects, all without departing from the invention. The present invention may be practiced without some or all of these specific details. In other instances, well known process operations have not been described in detail, in order not to unnecessarily obscure the present invention. Accordingly, the description is to be regarded as illustrative in nature, and not as restrictive.

### BRIEF DESCRIPTION OF THE FIGURES

FIG. 1 presents Table 1, which provides a set of human genetic markers useful for determining IFN responsiveness.

FIGS. 2A and 2B present examples of genes characterized by “wavy” pattern of expression during antiviral treatment. 2A: expression of STAT2 gene in whole cohort of treatment-naïve patients (genotype 1). 2B: expression of NUB1 gene in previously treated patients (genotype 1).

FIGS. 3A and 3B present examples of genes that maintained their expression levels throughout the start of treatment through Week 8. 3A: expression of IFNAR1 gene in whole cohort of treament-naïve patients. 3B: expression of SHFM1 gene in previously treated patients (genotype 1).

### DETAILED DESCRIPTION OF THE INVENTION

The subject matter of the disclosure relates to sets of genetic markers whose expression patterns correlate with important characteristics of response to hepatitis C specific treatment of individuals infected with HCV. More specifically, the subject matter of the disclosure provides for sets of genetic markers that can distinguish between patients that respond to hepatitis C specific therapy (Sustained Viral Responders or SVR) and patients that do not respond to hepatitis C specific therapy (Non-Responders or NR). Methods are provided for use of these markers to distinguish between these patient groups, and to determine general courses of treatment.

Prior to describing the subject matter of the disclosure in greater detail, the following terms are defined:

“Hepatitis C treatment-naïve patient” as the term is used herein refers to a patient having an HCV infection with viremia (HCV RNA measured by PCR) and who have not been previously treated with an interferon-based regimen and who are being scheduled to initiate treatment with pegylated interferon and ribavirin.

The term “hepatitis C treatment non responder patient” as used herein refers to a patient having a HCV infection with viremia (HCV RNA measured by PCR) and who has previously been treated and did not have sustained virologic response and who is being scheduled to initiate treatment with pegylated interferon and ribavirin.

“Sustained virologic response” as the term is used herein refers to undetectable HCV levels for 6 months after completion of therapy, as the definition of a “cure” for the infection.

“Responder phenotype” refers to the phenotype of an HCV infected patient that responds to the normal course of hepatitis C specific treatment such that at the end of a 24-week follow up period the patient does not have any detectable HCV, i.e., shows SVR.

“Non-responder phenotype” refers to the phenotype of an HCV infected patient that does not respond to the normal course of hepatitis C specific treatment such that during treatment or at the end of the 24-week follow up period, the patient has detectable virus, i.e., no SVR.

Classification of a sample as “responder phenotype” or “non-responder phenotype” is accomplished substantially as for the diagnostic markers described herein, wherein a template is generated to which the marker expression levels in the sample are compared. Where a set of markers has been identified that corresponds to two or more phenotypes, the marker sets can be used to distinguish these phenotypes. For example, the phenotypes maybe the diagnosis and/or prognosis of clinical states or phenotypes associated with other disease conditions, or other physiological conditions, wherein the expression level data is derived from a set of genes correlated with the particular physiological or disease condition.

The term “biological sample” as used herein refers to an bodily fluid or tissues sample likely to contain cells infected with HCV. Preferably, the biological sample is blood, most preferably peripheral blood lymphocytes. The sample may be collected in any clinically acceptable manner, but must be collected such that marker-derived polynucleotides (i.e., RNA) are preserved.

The terms “isolated,” “purified,” or “biologically pure” refer to material that is substantially or essentially free from components that normally accompany it as found in its native state. Purity and homogeneity are typically determined using analytical chemistry techniques such as polyacrylamide gel electrophoresis or high performance liquid chromatography. A protein or nucleic acid that is the predominant species present in a preparation is substantially purified. The term “purified” denoted that a nucleic acid or protein gives rise to essentially one band in an electrophoretic gel. Particularly, it means that the nucleic acid or protein is at least 85% pure, more preferably at least 95% pure, and most preferably at least 99% pure.

Methods for preparing total and poly (A)+ RNA are well known and are described generally in Sambrook et at., MOLECULAR CLONING—A LABORATORY MANUAL (2ND ED.), Vols. 1-3, Cold Spring Harbor Laboratory, Cold Spring Harbor, N.Y. (1989) which is hereby incorporated by reference in it's entirety and Ausubel et al., Current Protocols in Molecular Biology vol. 2, Current Protocols Publishing, New York (1994), which is hereby incorporated by reference in it's entirety. RNA may be isolated by the use of commercially available kits such as the RNeasy mini kit (Qiagen). RNA may be isolated from eukaryotic cells by procedures that involve lysis of the cells and denaturation of the proteins contained therein. RNA may be isolated from formalin-fixed paraffin-embedded using techniques well known in the art. Commercial kits for this purpose may be obtained from Zymo Research, Ambion, Qiagen, or Stratagene.

Additional steps may be employed to remove DNA. Cell lysis may be accomplished with a nonionic detergent, followed by micro-centrifugation to remove the nuclei and hence the bulk of the cellular DNA. In one embodiment, RNA is extracted from cells of the various types of interest using guanidinium thiocyanate lysis followed by CsCl centrifugation to separate the RNA from DNA (Chirgwin et at, Biochemistry 18:5294-5299 (1979)). Poly (A)+ RNA is selected by selection with oligo-dT cellulose (see Sambrook et al, MOLECULAR CLONING—A LABORATORY MANUAL (2ND ED.), Vols. 1-3, Cold Spring Harbor Laboratory, Cold Spring Harbor, N.Y. (1989), which is hereby incorporated by reference in it's entirety. Alternatively, separation of RNA from DNA can be accomplished by organic extraction, for example, with hot phenol or phenol/chloroform/isoamyl alcohol.

If desired, RNase inhibitors may be added to the lysis buffer. Likewise, for certain cell types, it may be desirable to add a protein denaturation/digestion step to the protocol. For many applications, it is desirable to preferentially enrich mRNA with respect to other cellular RNAs, such as transfer RNA (tRNA) and ribosomal RNA (rRNA). Most mRNAs contain a poly (A) tail at their 3′ end. This allows them to be enriched by affinity chromatography, for example, using oligo (dT) or poly (U) coupled to a solid support, such as cellulose or Sephadex (see Ausubel et al., CURRENT PROTOCOLS IN MOLECULAR BIOLOGY, vol. 2, Current Protocols Publishing, New York (1994). Once bound, poly (A)+ mRNA is eluted from the affinity column using 2 mM EDTA/0.1% SDS.

The sample of total RNA can comprise a plurality of different mRNA molecules, each different mRNA molecule having a different nucleotide sequence. In a specific embodiment, the mRNA molecules in the RNA sample comprise at least 100 different nucleotide sequences. More preferably, the mRNA molecules of the RNA sample comprise mRNA molecules corresponding to each of the marker genes. In another specific embodiment, the RNA sample is a human RNA sample. Preferably, the total mRNA is reverse transcribed into cDNA according to procedures well known in the art. In the present disclosure, target polynucleotide molecules are isolated from the biological sample taken from a patient who has or has had an HCV infection. mRNA or nucleic acids derived therefrom obtained from the sample may then be analyzed further. For example pairs of oligonucleotides specific for a marker or a set of gene markers (i.e. the markers presented in Table 1 may be used to amplify the specific message(s) in the sample. The amount of each message can then be determined or profiled and the correlation with a disease prognosis or probable response to a treatment regime is made.

“Marker” means an entire gene or portion thereof, or an EST derived from that gene, the expression level of which changes between certain conditions. Where the expression of the gene correlates with a certain condition, the gene is a marker for that condition.

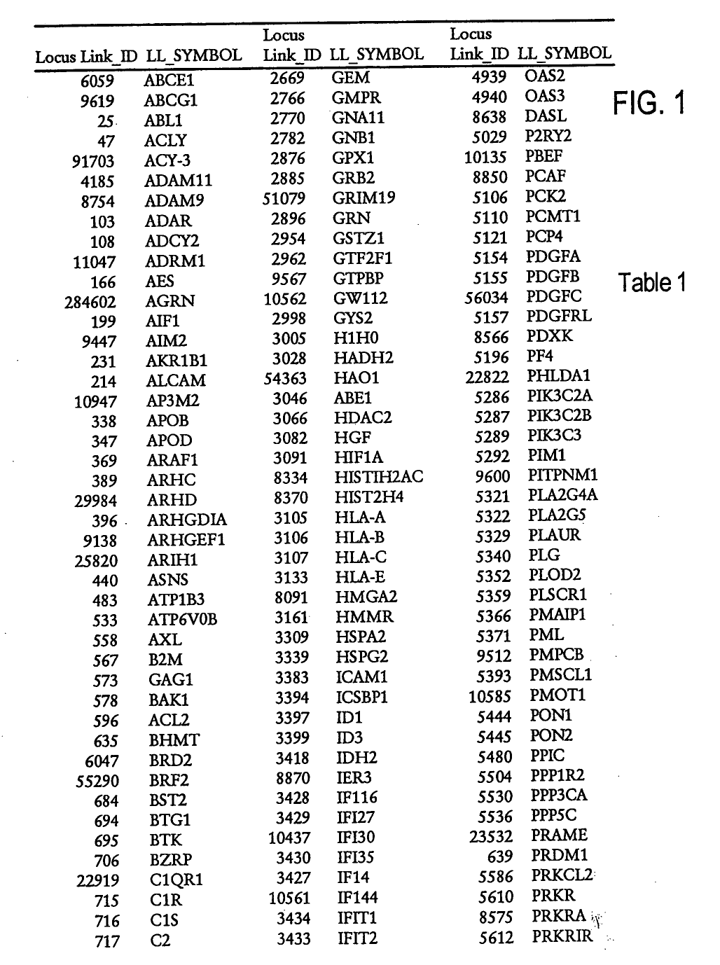

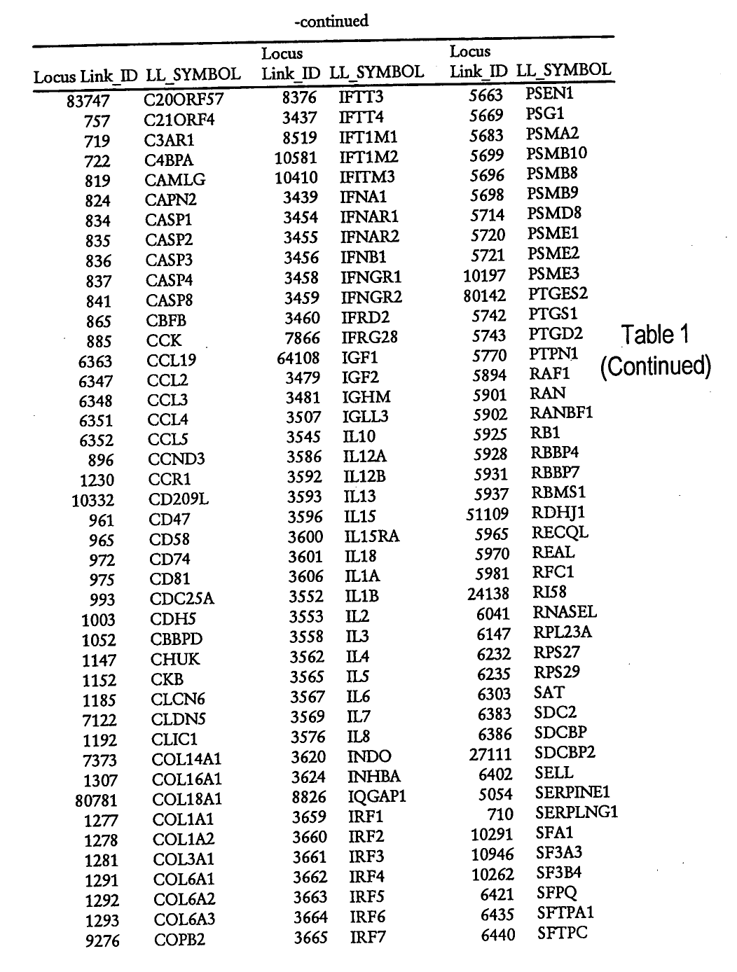

The marker genes of the invention are known human genes and are those described in Table 1 (see FIG. 1. and U.S. Publication No. US20050282179 which is hereby incorporated by reference in its entirety). Markers useful for determining IFN responsiveness, i.e. responder and non-responder phenotypes. Columns 1, 3, and 5 provide the Locus Link accession number for each marker gene. Columns 2, 4, and 6 provide the gene symbol for each of the respective marker genes listed in columns 1, 3, and 5. Locus Link is a web site maintained by the National Center for Biotechnology Information (NCBI). Locus Link provides information about the marker gene and its encoded protein of interest including links the GenBank accession numbers that provide both the nucleic acid and amino acid sequences of the marker gene and encoded protein. Based on this information, one of skill in the art would be able to devise and construct primers and probes to analyze the expression of each marker gene of interest in a sample obtained from a patient in accordance with the invention described herein.

“Marker-derived polynucleotide” means the RNA transcribed from a marker gene, any cDNA or mRNA produced therefrom, and any nucleic acid derived therefrom, such as synthetic nucleic acids having a sequence derived from the gene corresponding to the marker gene.

The expression level of a marker gene may be determined by isolating and determining the level (i.e., amount) of mRNA transcribed from each marker gene. Alternatively, or additionally, the level of specific proteins translated from mRNA transcribed from a marker gene may be determined.

“A plurality of marker genes” as used herein means two or more marker genes, preferably three or more marker genes, more preferably six or more marker genes, and most preferably 10 or more maker genes.

The level of expression of specific marker genes can be accomplished by determining the amount of mRNA, or polynucleotides derived therefrom, present in a sample. Although RT-PCR is preferred, any method for determining RNA levels can be used. For example, RNA is isolated from a sample and separated on an agarose gel. The separated RNA is then transferred to a solid support, such as a filter. Nucleic acid probes representing one or more markers are then hybridized to the filter by northern hybridization, and the amount of marker-derived RNA is determined. Such determination can be visual, or machine-aided, for example, by use of a densitometer. Another method of determining RNA levels is by use of a dot-blot or a slot-blot. In this method, RNA, or nucleic acid derived therefrom, from a sample is labeled. The RNA or nucleic acid derived therefrom is then hybridized to a filter containing oligonucleotides derived from one or more marker genes, wherein the oligonucleotides are placed upon the filter at discrete, easily-identifiable locations. Hybridization, or lack thereof, of the labeled RNA to the filter-bound oligonucleotides is determined visually or by densitometer. Polynucleotides can be labeled using a radiolabel or a fluorescent (i.e., visible) label. These examples are not intended to be limiting; other methods of determining RNA abundance are known in the art.

The level of expression of particular marker genes may also be assessed by determining the level of the specific protein expressed from the marker genes. This can be accomplished, for example, by separation of proteins from a sample on a polyacrylamide gel, followed by identification of specific marker-derived proteins using antibodies in a western blot. Alternatively, proteins can be separated by two-dimensional gel electrophoresis systems. Two-dimensional gel electrophoresis is well known in the art and typically involves isoelectric focusing along a first dimension followed by SDS-PAGE electrophoresis along a second dimension. See, e.g., Hames et al, 1990, GEL ELECTROPHORESIS OF PROTEINS: A PRACTICAL APPROACH, IRL Press, New York; Shevehenko et al., Proc. Nat Acad. Sci. USA 93:1440-1445 (1996); Saglioeco et al., Yeast 12:1519-1533 (1996); Lander, Science 274:536-539 (1996). The resulting electropherograms can be analyzed by numerous techniques, including mass spectrometry techniques, western blotting and immunoblot analysis using polyclonal and monoclonal antibodies.

Alternatively, marker-derived protein levels can be determined by constructing an antibody microarray in which binding sites comprise immobilized, preferably monoclonal, antibodies specific to a plurality of protein species encoded by the cell genome. Preferably, antibodies are present for a substantial fraction of the marker-derived proteins of interest. Methods for making monoclonal antibodies are well known (see, e.g., Harlow and Lane, 1988, ANTIBODIES: A LABORATORY MANUAL, Cold Spring Harbor, N.Y., which is incorporated in its entirety for all purposes). In one embodiment, monoclonal antibodies are raised against synthetic peptide fragments designed based on genomic sequence of the cell. With such an antibody array, proteins from the cell are contacted to the array, and their binding is assayed with assays known in the art. Generally, the expression, and the level of expression, of proteins of diagnostic or prognostic interest can be detected through immunohistochemical staining of tissue slices or sections.

Expression of marker genes in a number of tissue specimens may also be characterized using a “tissue array” (Kononen et al., Nat. Med 4(7):844-7 (1998)). In a tissue array, multiple tissue samples are assessed on the same microarray. The arrays allow in situ detection of RNA and protein levels; consecutive sections allow the analysis of multiple samples simultaneously.

Preferably, “determining an amount of a marker gene mRNA” refers to quantifying the expression level of a marker gene with the aim of determining whether the gene is expressed at a level that corresponds to a responder or non-responder phenotype. This can be done my methods commonly used in the art.

In one preferred embodiment, polynucleotide microarrays are used to measure expression so that the expression status of each of the markers above is assessed simultaneously. In a specific embodiment, oligonucleotide or cDNA arrays comprising probes hybridizable to the genes corresponding to each of the marker sets described above (i.e., markers to distinguish patients with good versus patients with poor prognosis). The microarrays may comprise probes hybridizable to the genes corresponding to markers listed in Table 1. For example, in a specific embodiment, the microarray is a screening or scanning array as described in Altschuler et al., International Publication WO 02/18646, published Mar. 7, 2002 and Scherer et al., International Publication WO 02/16650, published Feb. 28, 2002. The scanning and screening arrays comprise regularly-spaced, positionally-addressable probes derived from genomic nucleic acid sequences, both expressed and unexpressed. Such arrays may comprise probes corresponding to a subset of, or all of, the markers listed in Tables 1, 3 or 4, or a subset thereof as described above, and can be used to monitor marker expression in the same way as a microarray containing only markers listed in Tables 1, 3 or 4.

In yet another specific embodiment, the microarray is a commercially available cDNA microarray that comprises at least 2-4 of the markers listed in Table 1. Preferably, a commercially-available cDNA microarray comprises all of the markers listed in Table 1. However, such a microarray may comprise 5, 10, 15, 25, 50, 100, 150, 200 or more of the markers in Table 1, up to the maximum number of markers in the Table.

As noted above, the “probe” to which a particular polynucleotide molecule specifically hybridizes contains a complementary genomic polynucleotide sequence. The probes of the microarray preferably consist of nucleotide sequences of no more than 1,000 nucleotides. In some embodiments, the probes of the array consist of nucleotide sequences of 10 to 1,000 nucleotides. In a preferred embodiment, the nucleotide sequences of the probes are in the range of 10-200 nucleotides in length and are genomic sequences of a species of organism, such that a plurality of different probes is present, with sequences complementary and thus capable of hybridizing to the genome of such a species of organism, sequentially tiled across all or a portion of such genome.

Primer sets used in RT-PCR amplification assays to specifically amplify a particular polynucleotide contain polynucleotides that are identical to or complementary to the first strand synthesized in a reverse transcriptase reaction using a specific mRNA as a template. Primers may be 8-50 or more nucleotides in length, preferably 10-30 nucleotides in length and more preferably 15-25 nucleotides in length. Primers in a set used to amplify a specific nucleotide sequence are usually spaced 10-1000 nucleotides apart on that sequence, preferably the primers are spaced 25-500 nucleotides apart and more preferably, 50-250 nucleotides apart.

PCR primers are preferably chosen based on a known sequence of the genome that will result in amplification of specific fragments of genomic DNA. Computer programs that are well known in the art are useful in the design of primers with the required specificity and optimal amplification properties, such as Oligo version 5.0 (National Biosciences). It will be apparent to one skilled in the art that controlled robotic systems are useful for isolating and amplifying nucleic acids. Probes and primers are preferably selected using an algorithm that takes into account binding energies, base composition, sequence complexity, cross-hybridization binding energies, and secondary structure (see Friend et at, International Patent Publication WO 01105935, published Jan. 25, 2001; Hughes et al, Nat. Biotech. 19:342-7 (2001)).

The polynucleotide molecules which may be analyzed by the present invention (the “target polynucleotide molecules”) may be from any clinically relevant source, but are expressed RNA or a nucleic acid derived there from (e.g., cDNA), including naturally occurring nucleic acid molecules, as well as synthetic nucleic acid molecules. In one embodiment, the target polynucleotide molecules comprise RNA, including, but by no means limited to, total cellular RNA, poly (A) messenger RNA (mRNA) or fraction thereof, cytoplasmic mRNA, or RNA transcribed from cDNA (i.e., cRNA; see, e.g., Linsley & Schelter, U.S. patent application Ser. No. 09/411,074, filed Oct. 4, 1999, or U.S. Pat. Nos. 5,545,522, 5,891,636, or 5,716,785). Methods for preparing total and poly (A) RNA are well known in the art, and are described generally, e.g., in Sambrook et al., MOLECULAR CLONING—A LABORATORY MANUAL (2ND ED.), Vols. 1-3, Cold Spring Harbor Laboratory, Cold Spring Harbor, N.Y. (1989).

As used herein, an “amplified polynucleotide” or “amplicon” of the invention is a marker-containing nucleic acid molecule whose amount has been increased at least two fold by an nucleic acid amplification method performed in vitro as compared to its starting amount in a test sample. In other preferred embodiments, an amplified polynucleotide is the result of at least ten fold, fifty fold, one hundred fold, one thousand fold, or even ten thousand fold increase as compared to its starting amount in a test sample. In a typical PCR amplification, a polynucleotide of interest is often amplified at least fifty thousand fold in amount over the unamplified genomic DNA, but the precise amount of amplification needed for an assay depends on the sensitivity of the subsequent detection method used.

Generally, an amplified polynucleotide is at least twenty nucleotides in length. More typically, an amplified polynucleotide is at least thirty nucleotides in length. In a preferred embodiment of the invention, an amplified polynucleotide is at least fifty nucleotides in length. In a more preferred embodiment of the invention, an amplified polynucleotide is at least one hundred nucleotides in length. While the total length of an amplified polynucleotide of the invention can be the entire marker gene of interest, an amplified product is typically no greater than about five hundred nucleotides in length and is preferably between 100 and 300 nucleotides in length.

Kinetic RT-PCR may be performed using a variety of probes, buffers and PCR machines. Approaches to RT-PCR are described by Mackay et al., Nucleic Acids Research Vol. 30:1292-1305 (2002) and Kang et al., Nucleic Acids Research Vol. 28n No 2,:1-8 (2000) each of which is incorporated by reference in their entirety.

The polymerase chain reaction (PCR) (Freymuth, F. et al., (1995) J. Clin. Microbiol., 33:3352-3355, Mullis, K. B. et al., (1987) Methods Enzymol., 155:335-350) has been used as the new gold standard for detecting a wide variety of templates across a range of scientific specialties, including virology. The method utilizes a pair of synthetic oligonucleotides or primers, each hybridizing to one strand of a double-stranded DNA (dsDNA) target, with the pair spanning a region that will be exponentially reproduced. The hybridized primer acts as a substrate for a DNA polymerase (most commonly derived from the hemophilic bacterium. Thermus aquaticus and called Taq), which creates a complementary strand via sequential addition of deoxynucleotides. The process can be summarized in three steps: (i) dsDNA separation at temperatures >90° C., (ii) primer annealing at 50-75° C., and (iii) optimal extension at 72-78° C. (FIG. 1A). The rate of temperature change or ramp rate, the length of the incubation at each temperature and the number of times each set of temperatures (or cycle) is repeated are controlled by a programmable thermal cycler. Current technologies have significantly shortened the ramp times using electronically controlled heating blocks or fan-forced heated air flows to moderate the reaction temperature. Consequently, PCR is displacing some of the gold standard cell culture and serological assays (Niubo, J. et al., (1994). J. Clin. Microbiol., 32:1119-1120). Existing combinations of PCR and detection assays (called ‘conventional PCR’ here) have been used to obtain quantitative data with promising results. However, these approaches have suffered from the laborious post-PCR handling steps required to evaluate the amplicon (Ouatelli, J. C. et al., (1989) Clin. Micrbiol. Rev., 2: 217-226).

Traditional detection of amplified DNA relies upon electrophoresis of the nucleic acids in the presence of ethidium bromide and visual or densitometric analysis of the resulting bands after irradiation by ultraviolet light (Kidd, I. M. et al., (2000) J. Virol. Methods, 87:177-1811). Southern blot detection of amplicon using hybridization with a labeled oligonucleotide probe is also time consuming and requires multiple PCR product handling steps, further risking a spread of amplicon throughout the laboratory (Holland, P. M. et al., (1991) Proc. Natl. Acad. Sci. USA, 88:7276-7280). Alternatively, PCR—ELISA may be used to capture amplicon onto a solid phase using biotin or digoxigenin-labeled primers, oligonucleotide probes (oligoprobes) or directly after incorporation of the digoxigenin into the amplicon (van der Vliet, G. M. E., et al., J. Clin. Microbiol., 31:665-670, Keller, O. H. et al., (1990) J. Clin. Microbiol., 28:1411-1416, Kemp, D. J. et al., (1990) Gene, 94:223-228, Kox, L. F. F. et al., (1996) J. Clin. Microbiol., 34:2117-2120, Dekoneoko, A. et al., (1997) Clin. Diag. Virol., 8:113-121, Watzinger, F. et al., (2001) Nucleic Acids Res., 29:e52.). Once captured, the amplicon can be detected using an enzyme-labeled avidin or anti-digoxigenin reporter molecule similar to a standard ELISA format.

The possibility that, in contrast to conventional assays, the detection of amplicon could be visualized as the amplification progressed was a welcome one (Lomeli, H. et al., (1989) Clin. Chem., 35:1826-1831). This approach has provided a great deal of insight into the kinetics of the reaction and it is the foundation of kinetic or ‘real-time’ PCR (FIG. 1B) (Holland, P. M. et al., (1991) Proc. Natl. Acad. Sci. USA, 88:7276-7280, Lee, L. O. et al., (1993) Nucleic Acids Res., 21:3761-3766, Livak, K. J. et al., (1995) PCR Methods Appl., 4:357-362, Heid, C. A. et al., (1996) Genome Res., 6:986-994, Gibson, U. E. M. et al., (1996) Genome Res., 6:995-1001). Real-time PCR has already proven itself valuable in laboratories around the globe, building on the enormous amount of data generated by conventional PCR assays.

The monitoring of accumulating amplicon in real time has been made possible by the labeling of primers, probes or amplicon with fluorogenic molecules. This chemistry has clear benefits over radiogenic oligoprobes that include an avoidance of radioactive emissions, ease of disposal and an extended shelf life (Matthews, J. A. et al., (1988) Anal. Biochem., 169:1-25).

The increased speed of real-time PCR is largely due to reduced cycle times, removal of post-PCR detection procedures and the use of fluorogenic labels and sensitive methods of detecting their emissions (Wittwer, C. T. et al., (1990) Anal. Biochem., 186:328-331, Wittwer, C. T. et al., (1997) Biotechniques, 22:176-181). The reduction in amplicon size generally recommended by the creators of commercial real-time assays may also play a role in this speed, however we have shown that decreased product size does not necessarily improve PCR efficiency (Nitsche, A et al., (2000) J. Clin. Microbiol., 38:2734-2737).

The term “amount of internal control mRNA present in the total mRNA” refers to determining the amount of gene expression of genes that are also referred to the in the art as “housekeeping genes.” As used herein, a “house keeping” gene or “internal control” is meant to include any constitutively or globally expressed gene whose presence enables an assessment of marker gene mRNA levels. Such an assessment comprises a determination of the overall constitutive level of gene transcription and a control for variations in RNA recovery. “House-keeping” genes or “internal controls” can include, but are not limited to the cyclophilin gene, b-actin gene, the transferrin receptor gene, GAPDH gene, and the like. Eads et al., Cancer Research 1999; 59:2302-2306.

For example, Table 2 provides locus link information for the preferred housekeeping genes used to determine the relative amount of expression of the marker genes presented in Table 1.

TABLE 2
Housekeeping genes used
in normalization assay

| LOCUS_ID | LL_Symbol |
|---|---|
| 269261 | RPL12 |
| 5501 | PPP1CC |
| 5499 | PPP1CA |
| 6168 | RPL37A |
| 9798 | KIAA0174 |
| 493488 | SNRP70 |

A “relative marker gene index” refers to a comparison of the expression of the marker gene with the expression level of at least one internal control gene. If a marker gene index is below a specific threshold, then that marker does not correlate with a specific disease state. Preferably, if the marker gene index is above a certain threshold, it correlates with SVR.

A “threshold level” as used herein, was determined by correlating a certain disease state with the level of expression of a certain marker gene. Preferably, this was done by standard multiple regression analysis techniques. Below is a description of how this may be accomplished for any particular marker gene. A marker may be selected based upon significant difference of expression in a sample as compared to a standard or control condition, e.g., SVR.

Selection may be made by calculation of the statistical significance (i.e., the p-value) of the correlation between the expression of the marker and the condition or indication. Preferably, both selection criteria are used. Thus, in one embodiment of the present invention, markers associated with hepatitis C specific therapeutic agent response are selected where the markers show both more than two-fold change (increase or decrease) in expression as compared to a standard, and the p-value for the correlation between the existence of viral load and the change in marker expression is no more than 0.01 (i.e., is statistically significant).

The expression of the identified IFN response-related markers is then used to differentiate patients into responder and non-responder phenotypes. In a specific embodiment by way of working examples, using a number of patient samples, markers are identified by calculation of correlation coefficients between the clinical category or clinical parameter(s) and the linear, logarithmic or any transform of the expression ratio across all samples for each individual gene.

Next, the significance of the correlation is calculated. This significance may be calculated by any statistical means by which such significance is calculated. In one method, a set of correlation data is generated using a Monte-Carlo technique to randomize the association between the expression difference of a particular marker and the clinical category. The frequency distribution of markers satisfying the criteria through calculation of correlation coefficients is compared to the number of markers satisfying the criteria in the data generated through the Monte-Carlo technique. The frequency distribution of markers satisfying the criteria in the Monte-Carlo runs is used to determine whether the number of markers selected by correlation with clinical data is significant. Alternatively, the significance of the correlation may be calculated using a semi-supervised principal component approach, a semi-supervised clustering approach, a nearest neighbor classifier approach, or a univariate analysis.

Once a marker set is identified, the markers may be rank-ordered in order of significance of discrimination. One means of rank ordering is by the amplitude of correlation between the change in gene expression of the marker and the specific condition being discriminated. Another preferred means is to use a statistical metric.

The rank-ordered marker set may be used to optimize the number of markers in the set used for discrimination. This is accomplished generally in a “leave one out” method as follows. In a first run, a subset of the markers from the top of the ranked list is used to generate a template, where out of X samples, X-1 are used to generate the template, and the status of the remaining sample is predicted. This process is repeated for every sample until every one of the X samples is predicted once. In a second run, additional markers are added, so that a template is now generated from 10 markers, and the outcome of the remaining sample is predicted. This process is repeated until the entire set of markers is used to generate the template. For each of the runs, type 1 error (false negative) and type 2 errors (false positive) are counted; the optimal number of markers is that number where the type 1 error rate, or type 2 error rate, or preferably the total of type 1 and type 2 error rate is lowest.

The present invention provides marker genes for the identification of conditions or indications associated with response to Hepatitis C specific treatment in HCV infected patients. In particular, the invention provides for markers that can differentiate between HCV infected patients that will likely respond to Hepatitis C specific treatment versus HCV infected patients that will less likely respond to Hepatitis C specific treatment.

“Hepatitis C treatment” as the term is used herein refers to interferon or ribivirin based treatments as commonly prescribed in the art. Preferably, the treatment is in the form of a “hepatitis C-specific therapeutic agent”. A hepatitis C-specific therapeutic agent has interferon and/or ribivirin. Preferably, it has both. The interferon can be naturally occurring interferon, pegylated interferon or otherwise modified interferon, e.g., fused with albumin. Interferon alfa-2a (Roferon-A; Hoffmann-La Roche), inteferon alpha-2b (Intron-A; Schering-Plough), interferon alfacon-1 (Infergen; Intermune), peginterferon alpha-2b (Peg-Intron; Schering-Plough) and peginterferon alpha-2a (Pegasys; Hoffmann-La Roche) are all envisaged for use in a hepatitis C specific therapeutic agent. Additional drugs such as amantadine (Symmetrel) may also be included in the hepatitis C-specific therapeutic agent. Most preferably, the hepatitis C-specific therapeutic agent is a combination of peginterferon and ribivirin.

In a preferred embodiment, RT-PCR is used to determine marker and housekeeping gene expression levels. Each marker corresponds to a gene in the human genome, i.e., such marker is identifiable as all or a portion of a gene.

In the preferred method of the invention, the level of expression of one or more a marker genes in a bodily or tissue sample or is analyzed. Generally, the level of expression of the marker gene is then compared or normalized to the simultaneously determined expression level(s) of one or more internal control gene(s). This comparison or normalization provides an index of expression which takes into account the amount of global transcription in the tissue as well total mRNA recovery. For an example of such a procedure see also, U.S. Pat. No. 7,132,238, which is hereby incorporated by reference in its entirety.

If the index of expression of the marker gene is above a certain threshold then the patient from which the sample was derived, then a hepatitis C-specific treatment is likely to result in SVR in that patient. If the expression of the marker gene is below a certain threshold then the patient from which the sample was derived, then a hepatitis C-specific treatment is not likely to result in SVR in that patient—and the patient should be spared further treatment.

In certain circumstances, whether or not a threshold level is associated with SVR may depend upon the time point at which a sample was taken. For example, the expression level of marker gene at a high level prior to treatment with a hepatitis C specific therapeutic agent may not correlate with SVR whereas, the same level of expression 24 hours or 7 days later may in fact correlate with SVR.

### EXAMPLE 1

### Patient Population for Enrollment in the Studies Described Herein

### Inclusion Criteria

All patients must have met the following criteria: Adult male or female, chronic hepatitis C patients aged >18 years and positive for serum HCV-RNA by PCR prior to start of PEG interferon and ribavirin; Compensated liver disease; HIV (−); HBsAntigen (−) within one year of enrollment into the study; No treatment with ribavirin or other anti-viral or immunomodulatory drug for a minimum of 30 days prior to screening; Liver biopsy within 24 months of screening.

### Exclusion Criteria

Any cause for the liver disease based on previous clinical diagnosis or biopsy (where applicable) other than chronic hepatitis C. Patients must have met the following exclusion criteria: Patients not candidates for PEG Interferon and Ribavirin; Any other conditions, which in the opinion of the investigator would make the patient unsuitable for enrollment, or which could interfere with the patient participating in and completing the protocol.

The treatment regimen: PEG interferon alpha-2b and ribavirin.

The Antiviral Regimen: All subjects ere treated with interferon-based therapy with or without ribavirin per the weight/dose criteria list below.

| Subject Weight | | Dose |
|---|---|---|
| <65 | kg | 800 mg/day |
| 65-85 | kg | 1,000 mg/day |
| >85 kg-105 kg | | 1,200 mg/day |
| >105 | kg | 1,400 mg/day |

Subjects with negative HCV RNA at week 24 completed a 48-week course of therapy. Subjects with positive HCV RNA at week 24 were discontinued.

### EXAMPLE 2

### Study Procedures

Clinical evaluations during treatment of chronic hepatitis C were performed in three different phases:

Phase I: Early Phase (Treatment Week 0-week 24): Subjects were evaluated in the standard fashion for monitoring treatment with PEG interferon alpha-2b and Ribavirin;

Phase II: Late Phase (Treatment Weeks 25-48): For those subjects with undetectable HCV RNA (<50 IU/mL) at week 24, the full course of therapy was extended to 48 weeks. These subjects had additional monitoring at weeks 36 and 48 of therapy (see Visit Schedule, Table A). For those with positive HCV RNA at week 24, treatment was stopped and follow up was completed per protocol.

Phase III: Follow-Up Phase (Follow-up Weeks 4, 12 and 24) Subjects who completed the full course of therapy through treatment week 48 had follow up as delineated in Table A. Subjects who discontinued treatment before treatment week 48 were seen at follow-up weeks 4, 12 and 24.

### Tests Obtained at Baseline

All the clinical data needed prior to study entry was obtained within 1 month of starting antiviral therapy. Subjects are also required to have a liver biopsy within 24 months of screening for the study. Liver biopsies were scored according to the modified Knodell Histologic Activity scoring system.

### Tests Obtained in the Treatment Period

All subjects were monitored during the treatment period according to the visit schedule.

### Tests Obtained in the Follow-Up Period

As is standard in the care of subjects treated with interferon for Hepatitis C during the 6 month follow-up, a liver profile and CBC was drawn at weeks 4, 12, and 24. An HCV RNA by PCR will be drawn at follow-up week 24.

### EXAMPLE 3

### Methods

For each patient at each timepoint, 2.5 ml of blood was collected into each of five PAXgene Blood RNA tubes (QIAGEN, Cat. No. 762115). A total of 12.5 ml of blood is required for each patient at each time point. After blood collection, PAXgene tubes were stored at 4° C., and sent to Celera Diagnostics weekly. Total RNA was extracted from the Peripheral Blood Lymphocytes (PBLs) in each sample weekly at Celera Diagnostics.

### Biomarker Analysis

Celera Diagnostics has established a panel of interferon pathway related genes based on review of the literature. For example, Stark et al. Ann. Rev. Biochem 67: 277 (1998) and Der et al, PNAS 95:15623 (1998) provide many examples of interferon regulated genes and pathways. A database (http://www.lerner.ccf.org/labs/williams/xchip-html.cgi) with information related to interferon-stimulated genes was also used. Using reference RNA samples, Celera Diagnostics has developed highly sensitive and quantitative assays to monitor messenger RNA (mRNA) levels from several hundred interferon—regulated genes. The methods used in the biomarker analysis are described in US20050282179 which is incorporated by reference in its entirety.

### Gene Expression

Upon extraction of the RNA from the four time-point samples for each patient, the expression level of IFN pathway related genes in peripheral blood cells will be profiled. After the response to Hepatitis C specific treatment is available for all patient samples, the patient's mRNA levels will be analyzed for correlation with the patient's response to interferon.

### Data Analysis

After the response to Hepatitis C specific treatment was available for all patient samples, the patient's mRNA levels were analyzed for correlation with the patient's response to interferon. Patients were categorized according their patterns of response to therapy (Non Responder=NR vs. Sustained Virologic Response=SVR). Clinical (i.e., history, BMI, HWR, laboratory data, liver biopsy studies) and biomarker data between these two categories of patients was compared using univariate analysis and multivariate regression analysis. Clinical and biomarker data were analyzed to identify independent genomic markers of fibrosis and patterns of response to treatment.

Continuous data was summarized by using mean, SD, and median. Categorical data was summarized as percentages. For cross-sectional data, analysis-of-variance techniques with multiple comparison tests were employed for continuous data. Chi-square and rank sum techniques were used for categorical, ordinal or non-normal continuous data. For longitudinal data, repeated measures analysis-of-variance with contrasts were conducted to study ALT and HCV RNA over time among responders and non-responders. Kaplan-Meier techniques using the log rank test were used to examine differences in progression of fibrosis. Unless otherwise stated, p<0.05 is considered statistically significant. For the analysis of the genomic data, the inventors correlated gene profile with fibrosis and performed data mining for the correlation of clinical data and genomic data and pattern recognition.

### EXAMPLE 4

### Biomarker Assays

Marker assays (DNA, RNA, and protein profiling as well as other HCV/fibrosis—related markers).

Gene Expression Biomarkers Predicting Response to Pegylated Interferon Alfa (PEG-IFN) and Ribavirin (RBV) in the Peripheral Blood of Treatment-Naïve Patients with Chronic Hepatitis C (CH-C)

Responsiveness to HCV therapy depends on viral and host factors. Methods: Thirty CH-C patients (naïve) were started on PEG-IFN+RBV (standard doses of PEG-IFN alpha 2a or 2b and RBV). Blood samples were collected prior to treatment, 1 day, 1 week, 4 weeks, and 8 weeks after treatment. Total RNA was extracted, quantified and used for one step RT-PCR to profile 317 mRNAs (160 genes consisting of interferon-inducible, interferon pathway, immune response, and housekeeping genes). Expression levels of mRNAs were normalized with 6 “housekeeping” genes and a reference RNA. Multiple regression and stepwise selection were performed to assess differences in gene expression at different time points and predictive performance was evaluated for each model. Results: Demographics: 47±7 years, 64% Male, 61% Caucasian & 64% genotype 1 (G1). Prior to treatment, SVR was predicted by expression of STAT6 and CCL3 genes (Model p-value=0.0063, AUC=0.826, Sensitivity=0.923, Specificity=0.600) for all patients. In G1, SVR was predicted by expression of EP300 and SOCS6 (Model p-value=0.001, AUC=0.940, Sensitivity=0.857, Specificity=0.917). At day 1, SVR for entire cohort was predicted by expression of IFN-dependent genes (IF135, IRF8, IL15RA, GTPBP2, BCL2) (Model p-value=0.0094, AUC=0.901, Sensitivity=0.846, Specificity=0.929) and by IL1B and ADAM9 in G1 (Model p-value=0.0091, AUC=0.909, Sensitivity=0.857, Specificity=0.909). At day 7, SVR for entire cohort was predicted by expression of IL10, IRF8 and HIF1A (Model p-value=0.0002, AUC=0.928, Sensitivity=0.769, Specificity=0.867), while for G1, SVR was predicted by expression of PRKRIR (Model p-value=0.009, AUC=0.845, Sensitivity=1.000, Specificity=0.750). At Day 28, SVR was predicted by expression of AIM2, IRF2, YARS, IFNAR1, IRF8, HIF1A, CREB1 and CD58 (Model p-value=4.482e-006, AUC=1.000, Sensitivity=1.000, Specificity=1.000) for all patients. For G1, SVR was predicted by expression of AIM2, PLAUR, CCL3, IRF8 and CD58 (Model p-value=0.00045, AUC=1.000, Sensitivity=1.000, Specificity=1.000). At day 56, SVR was predicted by expression of PRKRIR, IRF5 and PSME2 (Model p-value=0.000065, AUC=0.959, Sensitivity=0.846, Specificity=1.000) for all patients and IRF4, IRF5, TRAF6, TAP1, IFNAR1 and PSMB9 in G1 (Model p-value=0.000015, AUC=1.000, Sensitivity=1.000, Specificity=1.000).

### EXAMPLE 5

### Gene Expression Biomarkers Predicting Response to Pegylated Interferon Alfa (PEG-IFN) and Ribavirin (RBV) in the Peripheral Blood of Patients with Chronic Hepatitis C (CH-C), Non-responder (NR) to Previous Treatment

Re-treatment of previously NR with another course of PEG-IFN and RBV is associated with low sustained virologic response (SVR). Methods: Thirty nine CH-C patients who had previously failed combination therapy were enrolled. Patients were being started on PEG-IFN+RBV (standard doses of PEF-IFN alpha 2a or 2b and RBV). Blood samples were collected into PAXgene™ RNA blood tubes (PreAnalytiX) prior to treatment, 1 day, 1 week, 4 weeks, and 8 weeks after the first dosing. Total RNA was extracted and quantified using RiboGreen™ Quantitation Kit (Molecular Probes). 5 ng of total RNA was used in each PCR reaction. One-step RT-PCR was used to profile 317 mRNAs (160 genes consisting of interferon-inducible, interferon pathway, immune response, and housekeeping genes) using Prism® 7900HT Sequence Detection System (Applied Biosystems). The expression levels of mRNAs of interest were normalized with 6 “housekeeping” genes. In addition, a Human Universal Reference RNA (Stratagene) was used as a control sample for further normalization. Patients who achieved week 12 and 24 virologic response received 48 weeks of therapy. All patients were followed for another 24 weeks to determine SVR. Gene expression profiles from different time points were compared to the baseline and associated with SVR. We performed multiple regression analysis and stepwise selection to assess differences in gene expression at different time points and predictive performance was evaluated for each model. Results: Demographic of patients included age: 49±6 years, 59% Male, 69% Caucasian, 78% HCV genotype 1, 16% G3 and 6% G4. In all patients prior to treatment, IRF2 expression level predicted SVR (Model p-value=0.04; AUC=0.718, Sensitivity=0.769, Specificity=0.611. Immediately after initiation of treatment, SVR in G1 was predicted by IFIT2 and JAK1 expression levels (Model p-value=0.0005; AUC=0.917, Sensitivity=1.000, Specificity=0.750). After a week of treatment, SVR in G1 patients was predicted by IRF4, BAG1, SOCS6, GMPR, LYN and SDCPB expression levels (Model p-value=0.0014; AUC=0.991, Sensitivity=1.000, Specificity=0.923), while for the entire CH-C cohort, it was predicted by NM1, PF4, BAG1, SOCS1, PDGFA and B2M expression levels (Model p-value<0.0014; AUC=0.989, Sensitivity=1.000, Specificity=0.929). At day 56, SVR in the entire CH-C cohort was predicted by the expression levels of NMI, IKBKB, RHOC, CD58 and PDGFA (Model p-value=0.004, AUC=0.917, Sensitivity=0.750, Specificity=1.000). On the other hand, in G1 patients, SVR was predicted by IL15 and COX17 expression levels (Model p-value=0.00034, AUC=0.962, Sensitivity=0.875, Specificity=1.000). Conclusions: A panel of non-invasive gene expression biomarkers is developed that can accurately predict SVR in NR CH-C patients.

### EXAMPLE 6

### Gene Expression Biomarkers can Predict Sustained Virologic Response (SVR) Early After Initiation of Pegylated Interferon Alfa (PEG-IFN) And Ribavirin (RBV) In Patients With Genotype 1 Chronic Hepatitis C (CH-C)

Responsiveness to HCV therapy depends on both viral and host factors. Patients with HCV genotype 1 (G1) have lower SVR rates. Determining rapid virologic response. (RVR) after 4 weeks of therapy and early virologic response (EVR) after 12 weeks of therapy can be helpful in the management of patients with CH-C. Nevertheless, an accurate biomarker to predict SVR early during the course of antiviral therapy is currently lacking. Methods: 44 CH-C patients with G1 (19 treatment-naïve and 25 non-responders (NR) to previous treatment) were started on PEG-IFN+RBV (standard doses of PEG-IFN alpha 2a or 2b and RBV). Blood samples were collected prior to treatment, 1 day, 1 week, 4 weeks, and 8 weeks after treatment. Treatment with antiviral therapy was continued for 48 weeks (if EVR was achieved and week-24 HCV RNA was undetectable). SVR was defined as undetectable HCV RNA 24 weeks after discontinuation of treatment. From the blood samples obtained at different time points, total RNA was extracted, quantified and used for one step RT-PCR to profile 317 mRNAs (160 genes consisting of interferon-inducible, interferon pathway, immune response, and housekeeping genes). Expression levels of mRNAs were normalized with 6 “housekeeping” genes and a reference RNA. Multiple regression and stepwise selection were performed to assess differences in gene expression at different time points and predictive performance was evaluated for each model. Results: Demographics: Patients were 49.11±6.96 years old with 54.5% males and 68.2% Caucasians. After 24 hours of antiviral treatment, SVR was predicted by IL1B and ADAM9 in G1-naïve patients (Model p-value=0.0091, AUC=0.909, Sensitivity=0.857, Specificity=0.909) and by IFIT2 and JAK1 expression levels in G1-NR patients (Model p-value=0.0005; AUC=0.917, Sensitivity=1.000, Specificity=0.750). After 7 days of antiviral treatment, SVR for G1-naïve patients was predicted by the expression of PRKRIR (Model p-value=0.009, AUC=0.845, Sensitivity=1.000, Specificity=0.750) and by the expression of IRF4, BAG1, SOCS6, GMPR, LYN and SDCPB in G1-NR patients (Model p-value=0.0014; AUC=0.991, Sensitivity=1.000, Specificity=0.923). Conclusions: A panel of non-invasive gene expression biomarkers is developed to predict SVR in G1 CH-C patients. This biomarker panel can become very useful during treatment of patients with HCV. These experiments show gene expression biomarkers predicting SVR early during anti-viral therapy of patients with HCV-G1.

### EXAMPLE 7

### Gene Expression Biomarkers Predicting Sustained Virologic Response in Patients with Chronic Hepatitis C and Treated with Pegylated Interferon-α and Ribavirin

Two groups of patients with chronic hepatitis C were enrolled in the study. The treatment naïve cohort included patients with chronic HCV and viremia who had not previously been treated (N=28). Another cohort included patients with chronic hepatitis C who had previously been treated and failed a course of combination therapy (N=31). Of these patients, 42% (13/31) had received at least 24 weeks of the optimal dose of combination therapy without virologic response, and 39% (12/31) had previously been treated with a suboptimal course of combination therapy without virologic response (requiring dose reduction of antiviral regimen or shortening duration of therapy due to side effects). An additional 19% (6/31) had relapsed after a complete course of combination therapy. For the purpose of this study, these patients were called the “previously treated cohort.”

Prior to being enrolled, informed consent was obtained from all patients, who then received a course of the standard doses of PEG-IFN α-2a or 2b and a weight-based dose of RBV. Patients with HCV genotype 1 who achieved EVR and were HCV RNA negative (<50 IU/mL after 24 weeks of therapy) were treated for a full 48 weeks. Patients with HCV genotype 2 and 3 were treated for only 24 weeks. Blood samples for mRNA profiling were collected in three PAXgene™ RNA blood tubes (PreAnalytiX) prior to the initiation of treatment as well as on days: 1, 7, 28, and 56 after treatment was initiated. Total RNA from each PAXgene™ RNA blood tube was extracted, pooled, and quantified with the RiboGreen™ Quantitation Kit (Invitrogen, Carlsbad, Calif.). The absence of genomic DNA in each RNA sample was confirmed by PCR assay with primers specific for human genomic DNA. Total RNA extracts were obtained at different time points and quantified. They were used for one step RT-PCR to profile 154 mRNAs reflecting expression of 153 human genes that belong to various IFN-inducible and immune responses related pathways along with a number of “housekeeping” genes. mRNA expression levels were normalized by using six housekeeping genes and a reference RNA. PCR primers were designed to amplify all known splice variants for a certain gene of interest or its specific variant according to annotation provided by Entrez Gene database of National Center of Biotechnology Information. Gene expression profiling was performed by using One-step RT-PCR with SYBR® Green and 5 ng of total RNA as a template. Other components of this PCR reaction have been described previously. Amplifications were performed in 384-well format with a duplicate of each 15 μl reaction using Prism® 7900HT Sequence Detection System (Applied Biosystems) and the cycle profile of 50° C. 2 min., 95° C. 1 min., and 60° C. 30 min., followed by 42 cycles of 95° C. 15 sec., and 60° C. 30 sec. ending with dissociation analysis. A total of 154 mRNA transcripts of interest (TOI) from 153 genes were profiled along with six housekeeping (HSKs) genes (PPP1CC, PPP1CA, RPL12, RPL37A, KIAA0174 and SNRP70). The relative gene expression levels were calculated by the ΔΔCt method. Briefly, the expression level of each gene was first normalized with the average of the expression levels of six housekeeping genes, and then further normalized to a control “calibrator,” Universal Human Reference RNA (Stratagene, La Jolla, Calif.). [ΔΔCt=ΔCt of sample (Ct of TOI−Average Ct of HSKs)−ΔCt of control (Ct of TOI−Average Ct of HSKs)]. The final ΔΔCt values of 148 mRNAs were used for statistical analyses. The positive and negative fold change values represent increased and decreased gene expression levels in patients who achieved SVR. Three series of data analysis included a first and second series that were performed separately for the treatment-naïve and previously treated cohorts, and a third series that was performed for the entire cohort (combining both treatment-naïve and previously treated cohorts). For each cohort, means and variances of gene expression level for each gene were calculated for each of the following groups:

All subjects with HCV genotype 1

All subjects with HCV non-genotype 1

All patients achieving SVR

All patients not achieving SVR

Only HCV genotype 1 patients achieving SVR

Only HCV genotype 1 patients not achieving SVR

HCV non-genotype 1 patients achieving SVR

HCV non-genotype 1 patients not achieving SVR.

For each of the three cohorts, three comparisons were performed for the expression levels of each gene quantified during each of the five visits (pre-treatment visit, and days 1, 7, 28, and 56 of the treatment). The following comparisons were made: A) In each cohort, all patients achieving SVR were compared to those not achieving SVR from the same cohort; B) HCV Genotype 1 patients from a cohort achieving SVR were compared to HCV Genotype 1 patients not achieving SVR; C) HCV Non-Genotype 1 from a cohort achieving SVR were compared to HCV Non-Genotype 1 not achieving SVR.

Mann-Whitney tests were performed for all the comparisons for each visit for all genes for all cohorts. Only p-values of <0.05 were considered significant (unless noted otherwise). Additionally, gene expression values were compared with the Significance Analysis of Microarrays (SAM) procedure, which computes a two-sample T-statistic for the normalized log ratios of gene expression levels for each gene. In the SAM analysis, each gene is assigned a numerical score (d) that is derived from the change in gene expression relative to the SD of repeated measurements across data sets. For genes with scores greater than an adjustable threshold, SAM uses permutations of the repeated measurements to estimate the percentage of genes identified by chance, the False Discovery Rate.

To detect genes with expression levels that were possibly associated with SVR, a series of multiple regression analyses with stepwise (bi-directional) selection of variables for all the cohorts and all the comparisons in each cohort and visit day was performed. The predictor variables were the genes that had been differentially expressed in patients achieving SVR in comparison with those with no SVR, and the dependent variable was the presence of SVR. The aim of the regression analysis was to assess whether the pattern of gene expression in peripheral blood cells is capable of predicting a sustained response to PEG-IFN+RBV treatment. Bi-directional selection began with a full model that contained all the genes with significantly different expression level between SVR and non-SVR patients (based on Mann-Whitney's outputs), and ended when no more improvement of the SVR fitting was available with addition or removal of gene predictors. Then, the predictive performance was evaluated for the generated models. Specifically, the sensitivity, specificity, and area under the ROC-curve (AUC) with 95% confidence intervals (CIs) were calculated for each model. All predictive models were then cross-validated using the leave-one-out (LOO) method. The regression analyses were executed with S-Plus 7.0 statistical package, and the ROC (receiver operating characteristic) analyses were generated using the MedCalc statistical tool.

### Gene Expression Activity Trends During Early Antiviral Therapy (Day 0 to Day 56)

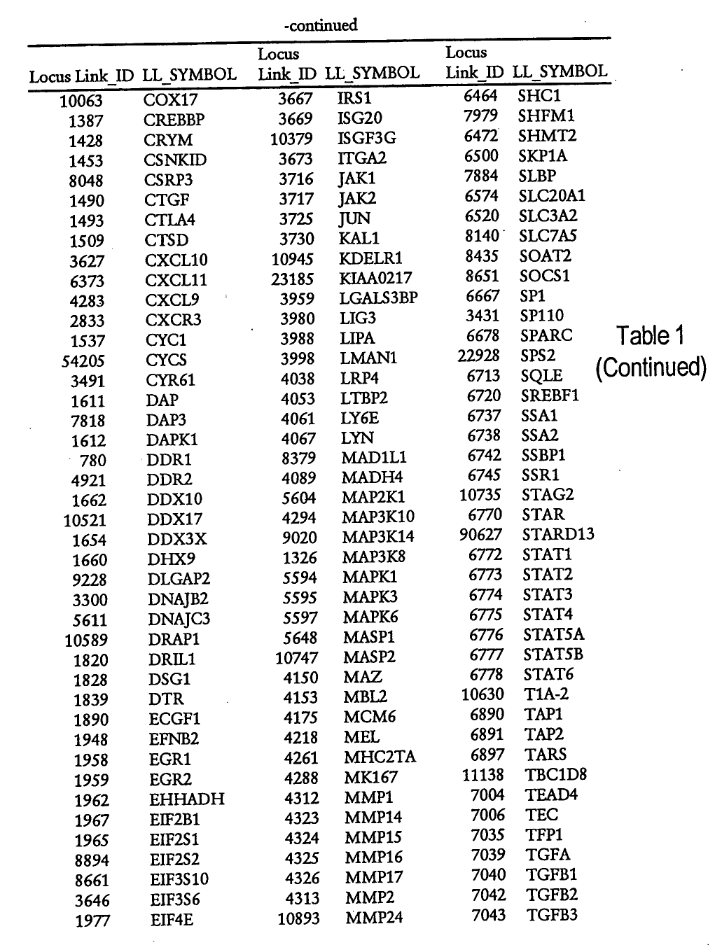

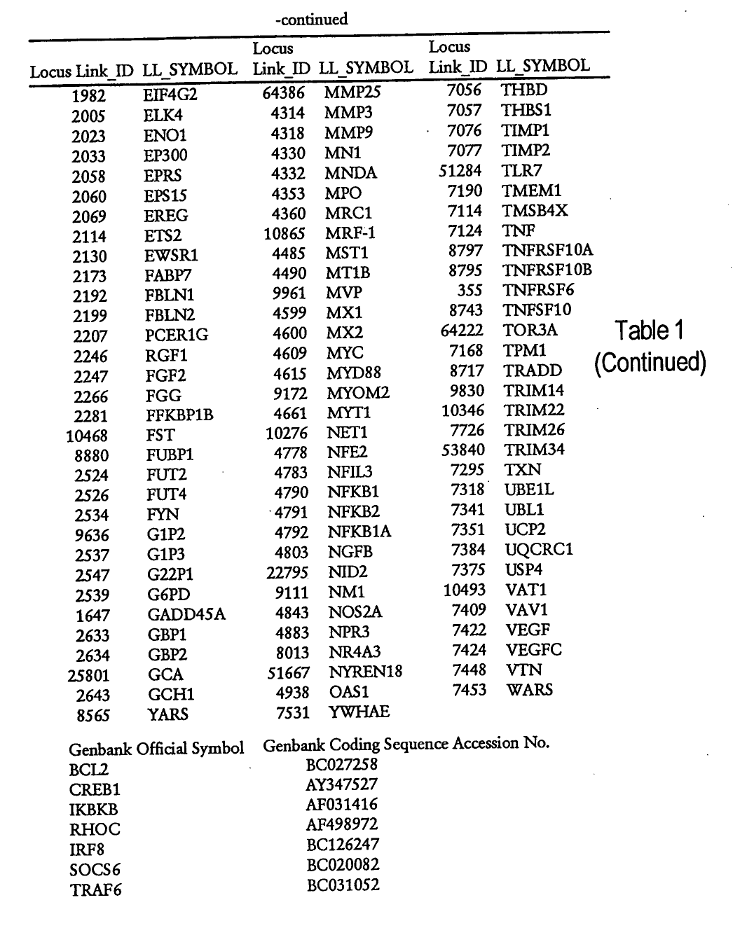

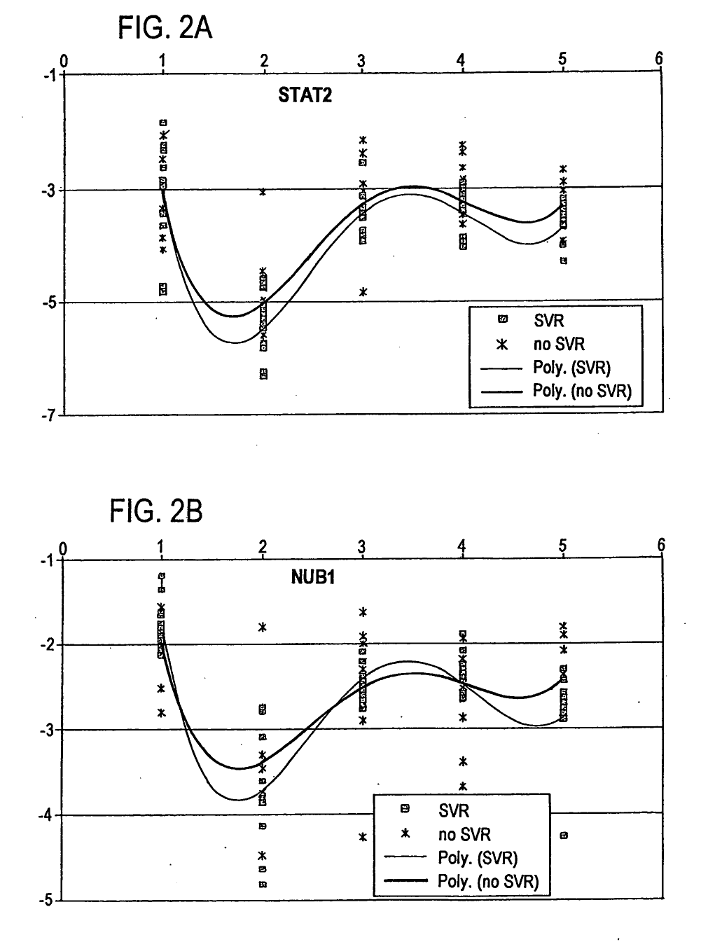

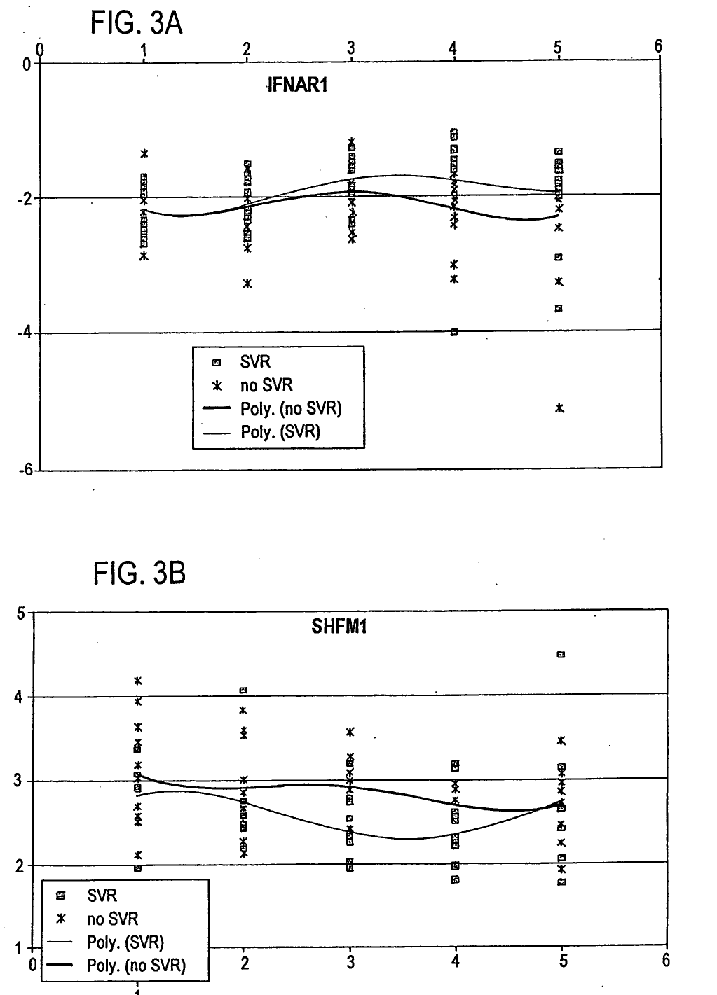

Considering the entire cohort (both naïve and previously treated), the activity of the profiled genes expression fluctuated at various time points during the treatment, reflecting relative down-regulation or up-regulation. Some genes underwent suppression after treatment was initiated and through 1 week of treatment, but their expression rebounded later during treatment, or vice versa, forming a characteristic “wavy” curve (FIGS. 2A and 2B). Other genes maintained their expression levels throughout the initiation of treatment through Week 8 (FIGS. 3A and 3B). Statistically significant genes contributors to the predictive models and passed Mann-Whitney cut-off of p-values <0.05 were found in both groups of these genes.

Differential gene expression associated with sustained virologic response in all naïve patients, naïve patients with HCV genotype 1, and naïve patients with non-genotype 1 were listed in Table 3. The genes that had been differentially expressed in patients achieving SVR in comparison with those with no SVR via leave-one-out validated models are underlined in Table 3.

TABLE 3
Genes with significant differential expression between SVR and no-SVR cohorts of
the treatment naïve patients. Genes used in leave-one-out validated models are underlined.

| | | |
|---|---|---|
| All (n = 28) | HCV Genotype 1 (n = 19) | HCV Non-G1 genotypes (n = 9) |

Pre-treatment

| | | | |
|---|---|---|---|
| (A) | STAT5A, STAT6, CCL3 | PRKRIR, STAT5A, EP300, CCL3, SOCS6 | CTLA4 |

24 hours after initiation of treatment

| | | | |
|---|---|---|---|
| (B) | IFI16, IFI30, IFI35, JAK2, IRF5, | IFI16, JAK2, STAT2, STAT3, IRF3, IRF5, | MX2, IFI30, IL10, PSMB8 |
| | SERPINE1, IL10, PSMB8, ICAM1, | IL10, ICAM1, PSME1, IL1B, LCK, | |
| | IRF8, PSME1, IL15RA, IL1B, NFIL3, | MMP9, ADAM9 | |
| | GTPBP2, ADAM9, BCL2 | | |

7 days after initiation of treatment

| | | | |
|---|---|---|---|
| (C) | PRKRIR, IRF5, IL10, IRF8, HIF1A, | PRKRIR, PSMB9, IRF8 | IRF8, STAT3, STAT5A, EP300, SP100, |
| | AP3M2 | | NUB1, CTNNB1, GBP2 |

28 days after initiation of treatment

| | | | |
|---|---|---|---|
| (D) | IFIT2, AIM2, IRF2, IRF5, PLAUR, | AIM2, IRF5, PLAUR, CCL3, FAS, IRF8, | IFI30, IFI35, ISG15, GBP1, JAK2, |
| | IL10, YARS, IFNAR1, FAS, IRF8, | HIF1A, CD58 | SERPINE1, TLR7, IL10, DHX9 |
| | PSME2, HIF1A, PTEN, CREB1, BTG1, | | |
| | CD58 | | |

56 days after initiation of treatment

| | | | |
|---|---|---|---|
| (E) | PRKRIR, STAT2, IRF5, TLR7, IL10, | PRKRIR, IRF2, IRF4, IRF5, TRAF6, | OAS1, OAS2, OAS3, OASL, MX1, |
| | PSME2 | TAP1, IFNAR1, PSMB9, PSME2, SELL, | IFI27, IFI30, IFI35, EIF2AK2, ISG15, |
| | | HIF1A | JAK2, STAT2, STAT4, ADAR, BST2, |
| | | | PLSCR1, SERPINE1, TLR7, IL10, TAP2, |
| | | | PSMB8, SOCS6, PSME1, PSME2, IL15, |
| | | | IL15RA, GTPBP2, GMPR, NUB1, LAP3, |
| | | | LGALS9, GPX1, ADAM9, PDGFA |

Considering the treatment-naïve patients, regardless of their genotypes, SVR was associated with higher pretreatment gene expression levels of cytoplasmic transcription factors STAT6 (1.19 fold) and STAT5A (1.24 fold), and a lower gene expression level of the cytokine CCL3 (−1.62 fold). This pattern of gene expression shifted to the expression of genes encoding several interferon-inducible proteins as well as cytokine encoding genes IL1B and IL10 shortly after treatment was initiated. The latter gene expression signatures persisted until late stages of the treatment (Day 28 and Day56).

Among the naïve patients infected with HCV non-G1 genotype, the strongest difference in expression levels was noted for cytotoxic T lymphocyte suppressing CTLA4 gene, which was over-expressed (−1.87 fold) in three patients who did not achieve SVR.

Models Predicting SVR in HCV Treatment Naïve Cohort

A multiple regression analysis and stepwise selection was used as well as the predictive performance of each model to assess whether PEG-IFN+RBV treatment response depends on the pattern of gene expression in the host (Table 4).

TABLE 4
Gene expression models predicting SVR in treatment-naïve patients

| | | | | | |
|---|---|---|---|---|---|
| | Gene(s) in the predictive | Model | Sensitivity, % | Specificity, % | |
| HCV Genotype | model | p-value | (95% CI) | (95% CI) | AUC |

Pre-treatment

| | | | | | |
|---|---|---|---|---|---|
| All | STAT6, CCL3 | 0.006252 | 69.2 | 60.0 | 0.774 |
| | | | (38.6-90.7) | (32.3-83.6) | (0.578-0.909) |
| Genotype 1 | EP300, SOCS6 | 0.001432 | 85.7 | 83.3 | 0.881 |
| | | | (42.2-97.6) | (51.6-97.4) | (0.651-0.980) |
| Non-G1 | CTLA4 | 0.018580 | 83.3 | 100.0 | 0.889 |
| genotypes | | | (36.1-97.2) | (30.5-100.0) | (0.517-0.982) |

24 hours after initiation of treatment

| | | | | | |
|---|---|---|---|---|---|
| All | IFI35, IRF8, IL15RA, | 0.009434 | 84.6 | 85.7 | 0.808 |
| | GTPBP2, BCL2 | | (54.5-97.6) | (57.2-97.8) | (0.611-0.932) |
| Genotype 1 | IL1B, ADAM9 | 0.009089 | 85.7 | 81.8 | 0.805 |
| | | | (42.2-97.6) | (48.2-97.2) | (0.554-0.949) |
| Non-G1 | PSMB8 | 0.014310 | 83.3 | 100.0 | 0.944 |
| genotypes | | | (36.1-97.2) | (30.5-100.0) | (0.585-0.965) |

7 days after initiation of treatment

| | | | | | |
|---|---|---|---|---|---|
| All | IL10, IRF8, HIF1A | 0.000201 | 76.9 | 73.3 | 0.892 |
| | | | (46.2-94.7) | (44.9-92.0) | (0.717-0.976) |
| Genotype 1 | PRKRIR | 0.009460 | 100.0 | 66.7 | 0.786 |
| | | | (58.9-100.0) | (34.9-89.9) | (0.540-0.936) |
| Non-G1 | GBP2, EP300, SP100, | 0.006501 | 100.0 | 100.0 | 1.000 |
| genotypes | NUB1, CTNNB1 | | (54.1-100.0) | (30.5-100.0) | (0.662-1.000) |

28 days after initiation of treatment

| | | | | | |
|---|---|---|---|---|---|
| All | AIM2, IRF2, YARS, | 0.000004 | 100.0 | 100.0 | 1.000 |
| | IFNAR1, IRF8, HIF1A, | | (75.1-100.0) | (78.0-100.0) | (0.875-1.000) |
| | CREB1, CD58 | | | | |
| Genotype 1 | AIM2, PLAUR, CCL3, | 0.000451 | 100.0 | 83.3 | 0.952 |
| | IRF8, CD58 | | (58.9-100.0) | (51.6-97.4) | (0.746-0.991) |
| Non-G1 | IFI30 | 0.001914 | 1.000 | 1.000 | 1.000 |
| genotypes | | | (54.1-100.0) | (30.5-100.0) | (0.662-1.000) |

56 days after initiation of treatment

| | | | | | |
|---|---|---|---|---|---|
| All | PRKRIR, IRF5, PSME2 | 0.000060 | 84.6 | 80.0 | 0.913 |
| | | | (54.5-97.6) | (51.9-95.4) | (0.743-0.984) |
| Genotype 1 | IRF4, IRF5, TRAF6, | 0.000105 | 85.7 | 100.0 | 0.964 |
| | TAP1, IFNAR1, PSMB9 | | (42.2-97.6) | (73.4-100.0) | (0.764-0.988) |
| Non-G1 | GMPR | 0.001347 | 100.0 | 100.0 | 1.000 |
| genotypes | | | (54.1-100.0) | (30.5-100.0) | (0.662-1.000) |

Note:
CI—confidence interval,
AUC—area under the ROC curve.

Gene Expression in the Pre-Treatment Samples Associated with SVR

When pre-treatment gene expression data from treatment-naïve patients with all HCV genotypes were analyzed together, SVR was associated with STAT6 and CCL3 gene expression levels (LOO-validated model p-value <0.0063, AUC=0.774, Sensitivity=0.692, Specificity=0.600). Limiting this analysis to 19 treatment-naïve HCV genotype 1 patients, SVR was associated with the expression of EP300 and SOCS6 (LOO validated model p-value<0.0015, AUC=0.881, Sensitivity=0.857, Specificity=0.833). Finally, considering 9 non-G1 treatment-naïve patients, SVR was associated with the expression of CTLA4 (LOO validated model p-value<0.02, AUC=0.889, Sensitivity=0.833, Specificity=1.000).

Gene Expression 24 Hours After Initiation of Treatment Associated with SVR:

Shortly after the initiation of the treatment (24 hours), SVR was associated with the expression of interferon-dependent genes and this dependence continued to be prominent throughout the treatment (Table 3 and 4). This pattern of gene expression generally persisted after 1 and 4 weeks of treatment.

Gene Expression 56 Days After Initiation of Treatment Associated with SVR

After 56 days of treatment for all naïve patients, SVR was associated with the expression of PRKRIR, IRF5 and PSME2 (LOO validated model p-value<0.00006, AUC=0.913, Sensitivity=0.846, Specificity=0.800). Limiting this analysis to 19 treatment-naïve HCV genotype 1 patients, the expression of IRF4, IRF5, TRAF6, TAP1, IFNAR1 and PSMB9 was associated with SVR (LOO validated model p-value=0.000105, AUC=0.964, Sensitivity=0.857, Specificity=1.000). On the other hand, in 9 treatment-naïve non-G1 HCV patients, SVR was associated with GMPR expression level (LOO validated model p-value<0.0013, AUC=1.000, Sensitivity=1.000, Specificity=1.000). The specificity, sensitivity, and AUC are depicted for each model at each time point in Table 4. The reliability of the models was lower for patients infected with HCV genotype 1, with the minimal AUC=0.786 (Day 7) increasing toward the later time points of the treatment. The model reached 100% of sensitivity and specificity at Day 56. Modeling that ignored the genotype of the virus resulted in the lowest reliability (AUC=0.774) in the pre-treatment model. As with the HCV genotype 1 models, the reliability of non-G1 genotypes models improved for the later stages of the treatment.

### Gene Expression Profiling of the Previously Treated Cohort

Gene Expression in the Pre-Treatment Samples Associated with SVR

Among all previously treated patients, regardless of their genotypes in the pretreatment samples, SVR was associated with lower levels of IFN regulatory factor (IRF2) expression, a negative regulator of IFN-α/β signaling (Table 5). The genes that had been differentially expressed in patients achieving SVR in comparison with those with no SVR via leave-one-out validated models are underlined in Table 5.

TABLE 5
Genes with significant differential expression between those achieving SVR and
those who did not achieve SVR in the previously treated cohort. Genes used in leave-one-out
validation models are underlined.

| | |
|---|---|
| All previously treated patients | HCV-G1 |
| (n = 31) | (n = 25) |

Pre-treatment

| | | |
|---|---|---|
| (A) | IRF2 | STAT1, AIM2, IRF2, RNASEL, SELL, HIF1A |

24 hours after initiation of treatment

| | | |
|---|---|---|
| (B) | NMI, TARS, LCK, AP3M2, BCL2 | JAK1, TARS |

7 days after initiation of treatment

| | | |
|---|---|---|
| (C) | IFITM2, PF4, SOCS1, SOCS6, IL15, SELL, GMPR, | IFITM2, PLAUR, RNASEL, PF4, TGFB1, BAG1, |
| | SHFM1, GPX1, PDGFA, B2M | SOCS1, SOCS6, IL1B, SELL, GMPR, LYN, MMP9, |
| | | SDCBP, SHFM1, GPX1, PTEN, CREB1, ATP6V0B, |
| | | PDGFA |

28 days after initiation of treatment

| | | |
|---|---|---|
| (D) | LIPA, SOCS6, MMP9, RHOC | LIPA, TAP2, MMP9, SHFM1 |

56 days after initiation of treatment

| | | |
|---|---|---|
| (E) | IFI30, NMI, PSMB8, PSME2, IL15, IKBKB, RHOC, | IFI6, TARS, IFNGR1, CCL4, CIITA, PSME1, PSME2, |
| | CD58, PDGFA, B2M | IL15, TANK, CD47, COX17, AIF1, RHOC, CD58, |
| | | B2M |

Gene Expression after Initiation of Treatment Associated with SVR

For the previously treated cohort at later stages of treatment, the gene expression profile shifted to the expression of genes encoding proteins unrelated to the interferon response. Instead, the expressed genes represented a variety of signaling cascades involved in the proliferation, apoptosis, and lymphocyte metabolism (Table 5).

### Models Predicting SVR in the Previously Treated Cohort

As with the treatment-naïve cohort, multiple regression analysis and stepwise selection were used to predict SVR in the previously treated cohort. The predictive performance was evaluated for each model (Table 6).

TABLE 6
Gene expression models predicting SVR in the previously treated patients. The
group of patients with HCV non-G1 genotypes was excluded from the analysis due to low
number of patients who did not achieve SVR.

| | | | | | |
|---|---|---|---|---|---|
| HCV | Gene(s) in the predictive | Model | Sensitivity, % | Specificity, % | |
| Genotype | model | p-value | (95% CI) | (95% CI) | AUC |

Pre-treatment

| | | | | | |
|---|---|---|---|---|---|
| All | IRF2 | 0.04747 | 69.2 | 58.8 | 0.624 |
| | | | (38.6-90.7) | (33.0-81.5) | (0.430-0.793) |
| Genotype 1 | IRF2, RNASEL, SELL | 0.02551 | 55.6 | 80.0 | 0.719 |
| | | | (21.4-86.0) | (51.9-95.4) | (0.500-0.880) |

24 hours after initiation of treatment

| | | | | | |
|---|---|---|---|---|---|
| All | NMI, TARS, AP3M2 | 0.02409 | 81.8 | 72.7 | 0.752 |
| | | | (48.2-97.2) | (39.1-93.7) | (0.524-0.908) |
| Genotype 1 | JAK1, TARS | 0.000603 | 87.5 | 100.0 | 0.958 |
| | | | (47.4-97.9) | (66.2-100.0) | (0.737-0.987) |

7 days after initiation of treatment

| | | | | | |
|---|---|---|---|---|---|
| All | PF4, SOCS1, GPX1, | 0.001165 | 76.9 | 92.3 | 0.817 |
| | PDGFA, B2M | | (46.2-94.7) | (63.9-98.7) | (0.617-0.939) |
| Genotype 1 | BAG1, MMP9 | 0.000192 | 80.0 | 100.0 | 0.909 |
| | | | (44.4-96.9) | (71.3-100.0) | (0.701-0.987) |

28 days after initiation of treatment

| | | | | | |
|---|---|---|---|---|---|
| All | LIPA, MMP9, RHOC | 0.001378 | 76.9 | 93.7 | 0.865 |
| | | | (46.2-94.7) | (69.7-99.0) | (0.687-0.962) |
| Genotype 1 | LIPA, TAP2, MMP9 | 0.005857 | 90.0 | 86.7 | 0.867 |
| | | | (55.5-98.3) | (59.5-98.0) | (0.671-0.967) |

56 days after initiation of treatment

| | | | | | |
|---|---|---|---|---|---|
| All | NMI, IKBKB, RHOC, | 0.006695 | 75.0 | 80.0 | 0.844 |
| | CD58, PDGFA | | (42.8-94.2) | (51.9-95.4) | (0.654-0.953) |
| Genotype 1 | IL15, COX17 | 0.000343 | 87.5 | 84.6 | 0.933 |
| | | | (47.4-97.9) | (54.5-97.6) | (0.733-0.991) |

When pre-treatment data was analyzed for the entire previously treated cohort (regardless of their genotypes), SVR was predicted by the expression of IRF2. When the cohort was limited to HCV genotype 1, addition of RNASEL and SELL genes improved AUC of the model (Genotype 1 group: LOO validated model p-value<0.026; AUC=0.719, Sensitivity=0.556, Specificity=0.800).

Performance of the SVR predictive models gradually increased after treatment was initiated. For example, in patients infected with HCV genotype 1 who were treated for 7 days, SVR was predicted by a combination of the expression levels of BAG1 and MMP9 (LOO validated model p-value<0.0002; AUC=0.909, Sensitivity=0.800, Specificity=1.000). At day 56 of treatment, SVR in genotype 1 patients was predicted by the expression of IL15 and COX17 (Model p-value=0.0003, AUC=0.933, Sensitivity=0.875, Specificity=0.846).
Models Predicting SVR in the Entire Cohort (Treatment Naïve and Previously Treated Groups)

When all the patients were analyzed regardless of their previous treatment experience, the pretreatment models predicting SVR were based mostly at expression levels of various intracellular signaling molecules (Table 7 and Table 8). On the other hand, both at early and later stages of the treatment, SVR was predicted by the expression of the effector genes, e.g., interleukins. As with treatment-naïve and previously treated cohorts, performance of the predictive models improved toward later stages of treatment. Before treatment, AUC of LOO validated model was 0.718, whereas at Day 56, a similarly calculated AUC was 0.853.

TABLE 7
Genes with significant differential expression between those achieving SVR and
those who did not achieve SVR using the entire cohort. Genes used in leave-one-out
validation models are underlined.

| | | |
|---|---|---|
| All (n = 59) | HCV Genotype 1 (n = 44) | HCV Non-G1 genotypes (n = 15) |

Pre-treatment

| | | |
|---|---|---|
| IFIT5, STAT6, SOCS1 | IFIT5, AIM2, IRF2, EP300, SELL, HIF1A | STAT6, IKBKG |

24 hours after initiation of treatment

| | | |
|---|---|---|
| IFI16, IFI30, IFI35, JAK2, LIPA, NMI, | LIPA, NMI, IRF5, IL1B, LCK, ADAM9, | PRKRIR, IL10 |
| IRF5, TLR4, IL10, TARS, PSMB8, | AP3M2, DHX9, BCL2 | |
| IRF8, PSME1, PSME2, IL15, IL1B, | | |
| LCK, TANK, CD47, ADAM9, AP3M2, | | |
| RHOC, BCL2 | | |

7 days after initiation of treatment

| | | |
|---|---|---|
| IFI30, IRF3, PLAUR, TLR7, IL10, | IRF3, IRF4, PLAUR, YARS, TARS, | IFI30, IL10, IL18 |
| YARS, TARS, BAG1, PSME2, IL15, | BAG1, SELL, GMPR, HIF1A, GPX1, | |
| SELL, GMPR, GPX1, CREB1, BTG1 | ATP6V0B, DHX9, PDGFA | |

28 days after initiation of treatment

| | | |
|---|---|---|
| LIPA, PLAUR, IL10, IFNAR1, SELL, | LIPA, IFR3, PLAUR, TRAF6, IFNAR1, | MX1, GBP1, JAK2, IL10, IL15, LAP3 |
| HIF1A, MMP9, FYN, PTEN, CREB1, | SOCS6, SELL, HIF1A, MMP9 | |
| BTG1 | | |

56 days after initiation of treatment

| | | |
|---|---|---|
| OAS3, IFI30, STAT2, BST2, IRF5, | PSME2, COX17 | OAS1, BST2, TLR7, TRIM14, IL10, |
| TLR7, IL10, CCL2, PSME2, IL18, | | PSMB8, CCL2, PSME2, GMPR, LAP3 |
| COX17, LAP3 | | |

TABLE 8
Gene expression models predicting SVR in the entire HCV cohort.

| | | | | | |
|---|---|---|---|---|---|
| | Gene(s) in the predictive | Model | Sensitivity, % | Specificity, % | |
| HCV Genotype | model | p-value | (95% CI) | (95% CI) | AUC |

Pre-treatment

| | | | | | |
|---|---|---|---|---|---|
| All | STAT6, SOCS1 | 0.001947 | 69.2 | 65.6 | 0.718 |
| | | | (48.2-85.6) | (46.8-81.4) | (0.584-0.828) |
| Genotype 1 | EP300, HIF1A | 0.002434 | 68.7 | 63.0 | 0.704 |
| | | | (41.4-88.9) | (42.4-80.6) | (0.545-0.833) |
| Non-G1 | STAT6 | 0.006893 | 90.0 | 80.0 | 0.840 |
| genotypes | | | (55.5-98.3) | (28.8-96.7) | (0.564-0.971) |

24 hours after initiation of treatment

| | | | | | |
|---|---|---|---|---|---|
| All | LIPA, NMI, RHOC, BCL2 | 0.0003142 | 70.8 | 84.0 | 0.808 |
| | | | (48.9-87.3) | (63.9-95.4) | (0.671-0.906) |
| Genotype 1 | LIPA, NMI, AP3M2, DHX9 | 0.0007532 | 66.7 | 95.0 | 0.837 |
| | | | (38.4-88.1) | (75.1-99.2) | (0.673-0.939) |
| Non-G1 | PRKRIR | 0.009655 | 88.9 | 80.0 | 0.844 |
| genotypes | | | (51.7-98.2) | (28.8-96.7) | (0.557-0.974) |

7 days after initiation of treatment

| | | | | | |
|---|---|---|---|---|---|
| All | PLAUR, IL10, BAG1 | 0.0000915 | 69.2 | 89.3 | 0.788 |
| | | | (48.2-85.6) | (71.7-97.6) | (0.656 to 0.888) |
| Genotype 1 | TARS, BAG1, HIF1A | 0.0001313 | 70.6 | 91.3 | 0.847 |
| | | | (44.1-89.6) | (71.9-98.7) | (0.697 to 0.940) |
| Non-G1 | IL10, IL18 | 0.0186 | 88.9 | 80.0 | 0.822 |
| genotypes | | | (51.7-98.2) | (28.8-96.7) | (0.532-0.966) |

28 days after initiation of treatment

| | | | | | |
|---|---|---|---|---|---|
| All | LIPA, IL10, MMP9, PLAUR | 0.0000226 | 92.3 | 61.3 | 0.844 |
| | | | (74.8-98.8) | (42.2-78.1) | (0.723-0.926) |
| Genotype 1 | LIPA, TRAF6, IFNAR1, | 0.001112 | 76.5 | 77.8 | 0.782 |
| | SOCS6, MMP9 | | (50.1-93.0) | (57.7-91.3) | (0.632-0.892) |
| Non-G1 | JAK2 | 0.007043 | 77.8 | 100.0 | 0.889 |
| genotypes | | | (40.1-96.5) | (40.2-100.0) | (0.596-0.986) |

56 days after initiation of treatment

| | | | | | |
|---|---|---|---|---|---|
| All | IRF5, PSME2 | 7.73e−006 | 76.0 | 83.3 | 0.853 |
| | | | (54.9-90.6) | (65.3-94.3) | (0.732-0.934) |
| Genotype 1 | PSME2 | 0.001189 | 80.0 | 80.0 | 0.829 |
| | | | (51.9-95.4) | (59.3-93.1) | (0.677-0.929) |
| Genotype 3 &4 | CCL2, GMPR | 0.002558 | 70.0 | 80.0 | 0.820 |
| | | | (34.8-93.0) | (28.8-96.7) | (0.541-0.963) |

In this disclosure there is described only the preferred embodiments of the invention and but a few examples of its versatility. It is to be understood that the invention is capable of use in various other combinations and environments and is capable of changes or modifications within the scope of the inventive concept as expressed herein. Thus, for example, those skilled in the art will recognize, or be able to ascertain, using no more than routine experimentation, numerous equivalents to the specific substances and procedures described herein. Such equivalents are considered to be within the scope of this invention. All references cited herein are incorporated by reference in their entirety for all purposes.

What is claimed is:

1. A method of treating hepatitis C in a hepatitis C treatment-naïve patient comprising:
a. obtaining a biological sample from the patient;
b. isolating total mRNA from the biological sample;
c. determining the relative marker gene index of said sample by measuring relative mRNA expression of a plurality of marker genes selected from the group consisting of those listed in Table 1; and
d. providing the patient with a hepatitis C-specific therapeutic agent when the relative marker gene index is above a threshold level that correlates with sustained viroloic response (SVR).

2. The method of claim 1 wherein the biological sample is obtained prior to the patient receiving a hepatitis C-specific therapeutic agent.

3. The method of claim 2 wherein the marker gene index is determined by measuring at least one of a gene selected from row A of Table 3.

4. The method of claim 1 wherein the biological sample is obtained about 24 hours after a treatment with a hepatitis C-specific therapeutic agent is initiated.

5. The method of claim 4 wherein the marker gene index is determined by measuring at least one of a gene selected from row B of Table 3.

6. The method of claim 1 wherein the biological sample is obtained about 7 days after a treatment with a hepatitis C-specific therapeutic agent is initiated.

7. The method of claim 6 wherein marker gene index is determined by measuring at least one of a gene selected from row C of Table 3.

8. The method of claim 1 wherein the biological sample is obtained about 28 days after a treatment with a hepatitis C-specific therapeutic agent is initiated.

9. The method of claim 8 the marker gene index is determined by measuring at least one of a gene selected from row D of Table 3.

10. The method of claim 1 wherein the biological sample is obtained about 56 days after a treatment with a hepatitis C-specific therapeutic agent is initiated.

11. The method of claim 10 the marker gene index is determined by measuring at least one of a gene selected from row E of Table 3.

12. The method of claim 1 wherein the internal control gene is selected from the group consisting of PPP1CC, PPP1CA, RPL12, RPL37A, KLAA0174 and SNRP70.

13. The method of claim 1 wherein the hepatitis C-specific therapeutic agent comprises ribivirin.

14. The method of claim 1 wherein the hepatitis C-specific therapeutic agent comprises an interferon.

15. A method of treating hepatitis C in a hepatitis C treatment non responder patient comprising:
a. obtaining a biological sample from the patient;
b. isolating total mRNA from the biological sample;
c. determining the relative marker gene index of said sample by measuring relative mRNA expression of a plurality of marker genes selected from the group consisting of those listed in Table 1;
d. providing the patient with a hepatitis C-specific therapeutic agent when the relative marker gene index is above a threshold level that correlates with sustained viroloic response (SVR).

16. The method of claim 15 wherein the biological sample is obtained prior to the patient receiving a renewed regimen of a hepatitis C-specific therapeutic agent.

17. The method of claim 16 wherein the marker gene index is determined by measuring at least one of a gene selected from row A of Table 5.

18. The method of claim 15 wherein the biological sample is obtained about 24 hours after a treatment with a renewed regimen of a hepatitis C-specific therapeutic agent is initiated.

19. The method of claim 18 wherein the marker gene index is determined by measuring at least one of a gene selected from row B of Table 5.

20. The method of claim 15 wherein the biological sample is obtained about 7 days after a treatment with a renewed regimen of a hepatitis C-specific therapeutic agent is initiated.

21. The method of claim 20 wherein the marker gene index is determined by measuring at least one of a gene selected from row C of Table 5.

22. The method of claim 15 wherein the biological sample is obtained about 28 days after a treatment with a renewed regimen of a hepatitis C-specific therapeutic agent is initiated.

23. The method of claim 22 wherein the marker gene index is determined by measuring at least one of a gene selected from row D of Table 5.

24. The method of claim 15 wherein the biological sample is obtained about 56 days after a treatment with a renewed regimen of a hepatitis C-specific therapeutic agent is initiated.

25. The method of claim 24 wherein the marker gene index is determined by measuring at least one of a gene selected from row E of Table 5.
